(19) United States
(12) Patent Application Publication
(10) Pub. No.: US 2024/0024645 A1
(43) Pub. Date: Jan. 25, 2024

(54) MEDICAL POWDER SPRAYING DEVICE WITH EASY CONTROL OF FLOW VOLUME OF POWDER BEING SPRAYED, AND OPERATING METHOD FOR POWDER SPRAYING DEVICE

(71) Applicant: THERACION BIOMEDICAL CO. LTD., Seongnam-si Gyeonggi-do (KR)

(72) Inventors: Eun Jin KIM, Seongnam-si, Gyeonggi-do (KR); Yong Suk CHOI, Gunpo-si, Gyeonggi-do (KR); Byung Nam KIM, Chungcheongbuk-do (KR)

(73) Assignee: THERACION BIOMEDICAL CO. LTD., Seongnam-si Gyeonggi-do (KR)

(21) Appl. No.: 18/025,262

(22) PCT Filed: Jan. 25, 2021
(86) PCT No.: PCT/KR2021/000953

§ 371 Date: Mar. 8, 2023

(30) Foreign Application Priority Data
Sep. 11, 2020 (KR) 10-2020-0117158

(51) Int. Cl. A61M 35/00; A61M 11/02; B05B 7/14

(52) U.S. Cl. CPC A61M 35/00; A61M 11/02; B05B 7/1404; A61M 2202/064; A61M 2205/3334

(57) ABSTRACT

A medical powder spraying device with easy control of the flow volume of the powder being sprayed and an operating method of the powder spraying device are disclosed. The powder spraying device is characterized by including a flow volume control means, and thus the flow volume of the powder being sprayed may be more easily controlled.

### TECHNICAL FIELD

The present invention relates to a medical powder spraying device with easy control of the flow volume of the powder being sprayed, and an operating method of the powder spraying device. More specifically, the powder spraying device is characterized by including a powder volume control means, and thus the flow volume of the powder being sprayed may be more easily controlled.

### BACKGROUND ART

Surgical operation refers to a medical practice in which a patient's skin or mucous membrane is incised and treated with a medical instrument. Depending on the severity of surgical operations, there are cases in which a patient's life is endangered and there is a risk of various unexpected accidents such as bleeding and adhesions. In order to prevent this, various products such as hemostatic agents, anti-adhesion agents, and wound dressings have been developed and used. In addition, these are often produced as powder for ease of use and management.

For example, bleeding means loss of blood out of blood vessels. The blood vessels deliver and circulate blood to whole body from the heart by pressure of itself. When a wound occurs in some blood vessels, a space through which the pressure escapes is generated and blood leaks out of the wound.

Hemostasis is a process to stop bleeding and maintaining normal state of blood flow. At the time of hemostasis, it is of utmost importance to quickly stop bleeding from a bleeding site by suturing or compressing a wound area with a hemostatic agent, bandage, or dressing. In particular, in recent years, the demand for hemostatic agents having excellent hemostatic ability while having biocompatibility is increasing. In order to respond to this, hemostatic agents using non-toxic biocompatible polymers as the main material are being developed to meet more improved hemostatic effect and various demands of the markets.

After tissue is damaged by bleeding, wounds, friction, etc. due to surgery or other reasons, blood coagulates in a healing process. Tissue adhesion refers to a phenomenon in which abnormal adhesion with surrounding tissues occurs during the coagulating process. Therefore, it is common to use an anti-adhesion agent in order to prevent this.

In addition, the wound refers to the destruction of the continuity of human tissue by external pressure or other factors. There is a risk of infection or inflammation due to exposure to an external environment during wound treatment. In order to prevent this, drugs such as wound dressing are produced and used.

However, even when a hemostatic agent, an anti-adhesion agent, and a wound dressing are developed for this purpose, it is also essential to apply or dose these agents in an appropriate amount at a target. For example, a material of a nozzle or a pipe for applying drugs may be different according to various purposes such as surgical operation, procedure, and laparoscopy, and a different length may be required. Therefore, a nozzle part for applying these agents needs to be frequently changed so as to be suitable for the purpose as necessary. Such replacement needs to be made quickly and simply. Accordingly, while the inventors of the present application were studying to solve the above problems, the inventors of the present application have developed a multi-purpose medical powder spraying device that allows a nozzle part to be replaced according to the purpose or the body part where medical powder is sprayed, so that an appropriate amount of medial powder is sprayed at a target, and applied for a patent (Patent Application No. 10-2020-0027493).

On the other hand, in recent years, polypectomy of the stomach or colon, mucosal resection for the treatment of early gastric cancer and colorectal cancer, and therapeutic endoscopic procedures have been mainly performed. Bleeding may occur during or after such procedures, and thus, emergency surgery or even death may occur.

Recently, hemostasis using an endoscope has been gradually used to treat bleeding during surgery, gastrointestinal bleeding, and bleeding occurring during collection of body tissues for biopsy. Hemostasis using the endoscope is a method of stopping bleeding by approaching a damaged mucous membrane to an endoscope conduit (catheter) inserted into the body and then spraying and dosing a powder formed hemostatic agent though the conduit.

Medical powder sprayers have been developed to achieve these purposes. In the same principle as paint spray, one of these medical powder sprayers operate to inject a hemostatic agent into a tube provided in the sprayer and then supply compressed air to the tube to spray and spread the hemostatic agent to a target area. However, since such powder sprayers cannot control the flow volume of the powder to be sprayed, the powder has to be sprayed only continuously. Therefore, there is an urgent need for a device capable of controlling the amount of powder to be sprayed according to the size or shape of wound.

While further researching to solve the above problems, the inventors of the present application have completed the present invention by discovering that the flow volume of sprayed powder could be more easily controlled when using a powder spraying device having an additional flow volume control means.

In this regard, Korean Patent Registration No. 10-0463141 discloses a medical powder for diagnosis, treatment, or drug delivery.

### DESCRIPTION OF EMBODIMENTS

### Technical Problem

The present invention has been made in an effort to solve the problems of the related art, and an object of the present invention is to provide a medical powder spraying device with a flow volume control means.

In addition, an object of the present invention is to provide an operating method of the medical powder spraying device.

### Solution to Problem

As a technical means for achieving the above technical challenges, an aspect of the present invention provides a powder spraying device (1).

The powder spraying device (1) includes: a powder container part 100 for containing powder; a first transfer pipe (120) having one side connected to the powder container part (100) to transfer the powder contained in the powder container part (100); a second transfer pipe (140) connected to the other side of the first transfer pipe (120) to transfer air and the powder transferred from the first transfer pipe (120); and a compressed air supply part (300) connected to one side of the second transfer pipe (140) to supply compressed air to the second transfer pipe (140), wherein the first transfer pipe (120) is provided with a flow volume control means (130) to control the flow volume of the powder transferred therein.

The flow volume control means (130) may include: an opening/closing means (135) for opening and closing the first transfer pipe (120); a piston (132) connected to one side of the opening/closing means (135) to perform a forward and backward reciprocating motion of the opening/closing means (135); and an elastic support (139) connected to the other side of the opening/closing means (135) to support the forward and backward reciprocating motion of the opening/closing means (135).

The opening/closing means (135) may have an opening hole (136) formed on one side thereinside.

The flow volume control means (130) may further include a frame (131) for supporting the first transfer pipe (120), the opening/closing means (135), the piston (132), and the elastic support (139).

The powder container part (100) may be formed in a funnel shape in which the diameter thereof decreases toward the first transfer pipe (120).

The powder spraying device (1) may further include an air pump switch (370) for operating the air supply part (300).

A vibrating means (200) may be attached to the outer surface of the first transfer pipe (120) or the second transfer pipe (140).

The powder spraying device (1) may further include a spraying means (400) connected to the other side of the second transfer pipe (140) to spray the transferred powder and the air to the outside.

The powder spraying device (1) may further include a trigger (500) for operating the flow volume control means (130).

The powder spraying device (1) may further include a power supply part (600) for operating the air supply part (300) and the flow volume control means (130).

In addition, another aspect of the present invention provides an operating method of the powder spraying device (1).

The operating method of the powder spraying device (1) includes: operating the air supply part (300) to supply air to the second transfer pipe (140); operating the flow volume control means (130) to supply the powder contained in the powder container part (100) to the first transfer pipe (120) and the second transfer pipe (130); and releasing the powder supplied to the second transfer pipe (130) to the outside together with the air.

### Advantageous Effects of Disclosure

Since a powder spraying device according to the present invention includes a funnel-shaped powder container part and a vibrating means, powder may be easily sprayed without clogging.

In addition, since the powder spraying device according to the present invention includes a flow volume control means to control the flow volume of the supplied powder, the spraying volume of the powder required during surgery may be easily controlled.

### BRIEF DESCRIPTION OF DRAWINGS

FIG. 1 is an overall diagram illustrating a powder spraying device according to an embodiment of the present invention.

FIG. 2 is an overall diagram illustrating a powder spraying device including a different type of powder container part according to an embodiment of the present invention.

FIG. 3 is an overall diagram illustrating a powder spraying device according to another embodiment of the present invention.

FIGS. 4 and 5 are schematic diagrams illustrating an operating principle of a flow volume control means according to an embodiment of the present invention.

### MODE FOR CARRYING OUT INVENTION

Hereinafter, the present invention will be described in more detail. However, the present invention may be embodied in various different forms and is not limited by embodiments described herein, and the present invention is only defined by the claims to be described below.

In addition, the terms as used herein are only used to describe specific embodiments, and are not intended to limit the present invention. The singular forms “a,” “an,” and “the” as used herein are intended to include the plural forms as well unless the context clearly indicates otherwise. In the specification of the present invention, the phrase “including a certain element” means “further including other elements” rather than excluding other elements unless otherwise stated.

A first aspect of the present application provides a powder spraying device (1).

The powder spraying device (1) includes: a powder container part (100) for containing powder; a first transfer pipe (120) having one side connected to the powder container part (100) to transfer the powder contained in the powder container part (100); a second transfer pipe (140) connected to the other side of the first transfer pipe (120) to transfer air and the powder transferred from the first transfer pipe (120); and an air supply part (300) connected to one side of the second transfer pipe (140) to supply air to the second transfer pipe (140), wherein the first transfer pipe (120) is provided with a flow volume control means (130) to control the flow volume of the powder transferred therein.

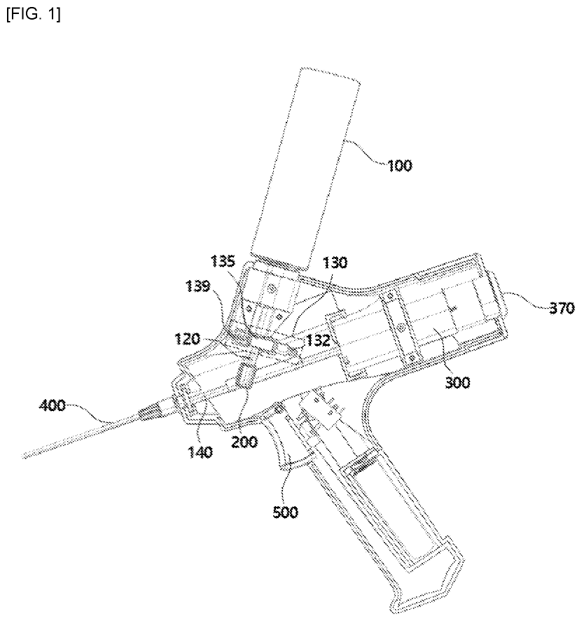

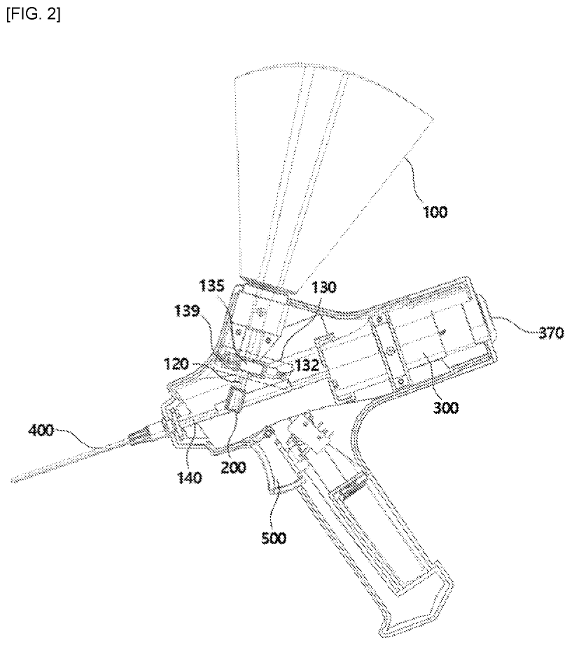

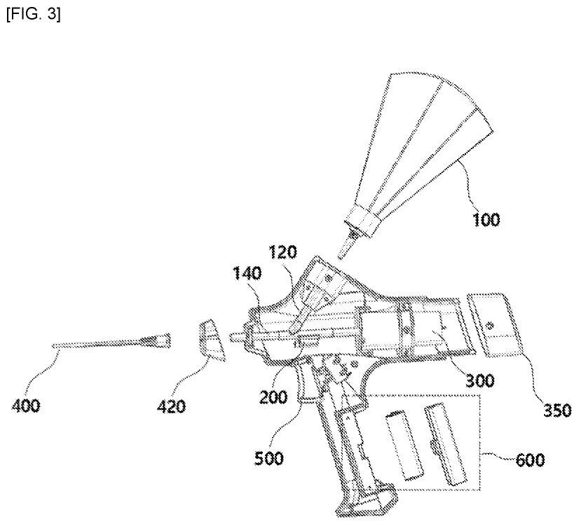

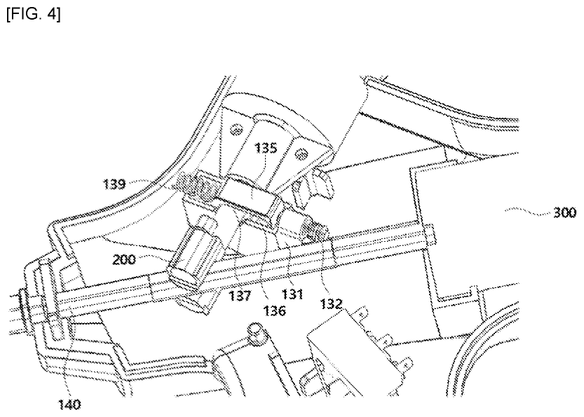

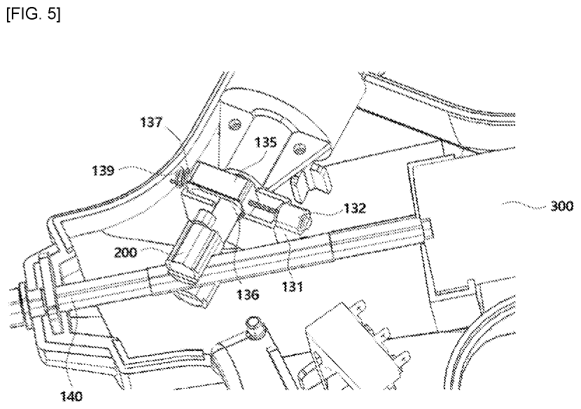

Hereinafter, a powder spraying device (1) according to a first aspect of the present application will be described in detail with reference to FIGS. 1 to 5. At this time, FIG. 1 is an overall diagram illustrating the powder spraying device (1), FIG. 2 is an overall diagram illustrating the powder spraying device (1) including a different type of powder container part (100), FIG. 3 is an overall diagram illustrating a powder spraying device (1) according to another embodiment, and FIGS. 4 and 5 are schematic diagrams illustrating an operating principle of a flow volume control means (130).

In an embodiment of the present application, the powder spraying device (1) may be used to treat bleeding during surgery. Specifically, the powder spraying device (1) may be used for hemostasis using an endoscope. That is, the powder spraying device (1) may be used in laparoscopy and may operate to spray and dose hemostasis powder along with compressed air to a damaged mucous membrane requiring hemostasis. To this end, it is essential to easily spray the powder without clogging. Therefore, the powder spraying device (1) according to the present invention may include elements to be described below.

In an embodiment of the present application, the powder spraying device (1) may include a powder container part (100) for containing powder. In this case, the powder container part (100) may be located at the upper end of the powder spraying device (1). As illustrated in FIGS. 2 and 3, the powder container part (100) may be formed in a funnel shape in which the diameter thereof decreases toward the first transfer pipe (120). That is, since the powder container part (100) has the above-described shape, the powder contained therein may be more easily transferred to the first transfer pipe (120) by gravity. Even when the amount of remaining powder is small, the powder may be smoothly transferred to the first transfer pipe (120). Therefore, it is very cost-effective. On the other hand, the size of the powder container part (100) is not particularly limited. However, when the size of the powder container part (100) is too small, the amount of powder contained in the powder container part (100) may be too small, which may cause inconvenience of having to fill the powder frequently. When the size of the container part (100) is too large, the volume of the powder spraying device (1) including the powder container part (100) may increase, which may cause difficulty in using the spraying device (1) for hemostasis. Therefore, the powder container part (100) may have to be formed in an appropriate size.

In an embodiment of the present application, the powder container part (100) may be formed detachably from the powder spraying device (1). To this end, the lower end of the powder container part (100) may be formed in a tubular shape having a constant diameter so as to be inserted into the first transfer pipe (120). That is, when the powder contained in the powder container part (100) is exhausted, the powder container part (100) may be detached from the powder spraying device (1) and may be filled again with powder. On the other hand, the powder container part (100) may include a lid that can be opened and closed on the upper surface. In this case, the lid may be opened to fill the power container part (100) with powder.

In an embodiment of the present application, the powder container part (100) may be preferably made of a flexible material. That is, since the powder container part (100) is made of a flexible material, it may be possible to increase the volume by a certain amount according to the content of the powder contained therein. Therefore, since the powder container part (100) made of a flexible material for the same volume contains more powder, the time of resupply of the powder may be increased, which may be more cost-effective.

In an embodiment of the present application, the powder spraying device (1) may include a first transfer pipe (120) having one side connected to the powder container part (100) to transfer the powder contained in the powder container part (100), and a second transfer pipe (140) connected to the other side of the first transfer pipe (120) to transfer air and the powder transferred from the first transfer pipe (120).

In an embodiment of the present application, as illustrated in FIGS. 1 to 3, the other side of the first transfer pipe (120) may be connected to the middle upper surface of the second transfer pipe (140), and the powder transferred to the first transfer pipe (120) by gravity may be naturally transferred to the second transfer pipe (140).

In an embodiment of the present application, a vibrating means (200) may be attached to the outer surface of the first transfer pipe (120), and a plurality of vibrating means (200) may be provided. More specifically, the vibrating means (200) may be attached to both sides of the first transfer pipe (120) in a direction in which the first transfer pipe (120) is formed from the position where the first transfer pipe (120) meets the second transfer pipe (140). At this time, as illustrated in FIGS. 3 and 4, the term “both sides” may refer to the front and rear sides with respect to an angle formed by the first transfer pipe (120) and the second transfer pipe (140). Therefore, since the vibrating means (200) is attached to the above-described position, the vibrating means (200) may not spatially overlap the first transfer pipe (120) and the second transfer pipe (140).

In an embodiment of the present application, the powder contained in the powder container part may be more easily sprayed to the outside by transmitting vibration generated from the vibration means (200) to the first transfer pipe (120). On the other hand, it may be designed to transmit vibration generated by the vibrating means (200) not only to the inside of the first transfer pipe (120) but also to the powder container part (100) and the second transfer pipe (140). To this end, the vibrating means (200) may use a vibrating motor to generate stronger vibration. That is, since the vibration generated through the vibrating means (200) is transmitted to the powder container part (100), the first transfer pipe (120), and the second transfer pipe (140), the powder accumulated or clogged inside may be smoothly transferred and sprayed to the outside.

In an embodiment of the present application, some of the plurality of vibrating means (200) may be preferably attached to the outer surface of the second transfer pipe (140). In this case, some of the plurality of vibrating means (200) may be installed behind a portion where the first transfer pipe (120) and the second transfer pipe (140) are in contact with each other, or may be attached to the lower outer surface of the second transfer pipe (140). At this time, the term “behind” refers to an opposite direction of a spraying means (400) to be described below. Since the vibrating means (200) is installed in the above-described position, the vibrating means (200) does not spatially overlap the first transfer pipe (120).

In an embodiment of the present application, the powder spraying device (1) may include the air supply part (300) connected to one side of the second transfer pipe (140) to supply air to the second transfer pipe (140).

In an embodiment of the present application, the air supply part (300) is a means for supplying air to the inside of the second transfer pipe (140), and may push the powder, which is transferred to the second transfer pipe (140), to the spraying means (400) to be described below. To this end, the air supplied from the air supply part (300) may have a constant pressure. For example, a piston or a diaphragm type air pump may be used.

In an embodiment of the present application, the air supply part (300) may further include a heating device (not illustrated) for removing moisture in the air supplied to the second transfer pipe (140). In this manner, it may be possible to prevent the powder transferred to the second transfer pipe (140) from being agglomerated due to moisture. For example, a semiconductor or a hot wire may be used. On the other hand, a cover (350) may be formed on the other side of the air supply part (300). The cover (350) may prevent the air supply part (300) and other connecting pipes from being separated to the outside of the powder spraying device (1).

In an embodiment of the present application, an air pump switch (370) may be installed in the cover (350). That is, the air supply part (300) may be operated by pushing the air pump switch (370).

In an embodiment of the present application, the powder spraying device (1) may include a spraying means (400) connected to the other side of the second transfer pipe (140) to spray the transferred powder and the air to the outside.

In an embodiment of the present application, the spraying means (400) may be connected to the powder spraying device (1) through the connecting means (420). In this case, the spraying means (400) may be formed in a tubular form so as to spray the powder and the air to the outside, and a connection port may be formed at a portion connected to the other side of the second transfer pipe (140). That is, the powder spraying device (1) may have a form in which the spraying means (400) is connected by the connection port and the connecting means (420) connected to the other side of the second transfer pipe (140).

In an embodiment of the present application, the connecting means (420) and the connection port may be screwed to each other. That is, the screwing form may enable easier and quicker connection than other connection methods. Accordingly, when the spraying means (400) is contaminated or damaged during use, the spraying means (400) may be quickly replaced with another spraying means (400).

In an embodiment of the present application, the pipe of the spraying means (400) may be made of a flexible material. Therefore, while spraying the powder to a target for hemostasis, the pipe may be bent even when the spraying means (400) comes into contact with internal tissue, thereby preventing possible rupture or damage of internal tissue.

In an embodiment of the present application, the first transfer pipe (120) may include a flow volume control means (130) to control the flow volume of the powder transferred therein.

In an embodiment of the present application, the flow volume control means (130) may include: an opening/closing means (135) for opening and closing the first transfer pipe (120); a piston (132) connected to one side of the opening/closing means (135) to perform the forward and backward reciprocating motion of the opening/closing means (135); and an elastic support (139) connected to the other side of the opening/closing means (135) to support the forward and backward reciprocating motion of the opening/closing means (135), and may include a frame (131) for supporting the first transfer pipe (120), the opening/closing means (135), the piston (132), and the elastic support (139).

In an embodiment of the present application, the opening/closing means (135) may have an opening hole (136) formed on one side thereinside, and the remaining portion may be closed. In the present specification, the closed portion will be referred to as a closing means (137). In this case, the diameter of the opening hole (136) may be the same as the inner diameter of the first transfer pipe (120). On the other hand, for smooth operation of the opening/closing means (135), the first transfer pipe (120) may be cut at a position where the opening/closing means (135) is located. That is, referring to FIGS. 4 and 5, the opening/closing means (135) may be inserted into the cut portion of the first transfer pipe (120), the powder may be supplied when the opening hole (136) meets the first transfer pipe (120), and the supply of the powder may be stopped when the closing means (137) meets with the first transfer pipe (120). That is, the powder may be supplied and stopped through the forward and backward reciprocating motion of the closing means (137).

In an embodiment of the present application, the piston (132) may be formed on one side of the opening/closing means (135) so as to perform the forward and backward reciprocating motion of the opening/closing means (135), and the elastic support (139) may be formed on the other side of the opening/closing means (135) so as to support the forward and backward reciprocating motion. That is, when the piston (132) operates to push the opening/closing means (135), the opening hole (136) may be positioned in the first transfer pipe (120) so that the powder is transferred. When the operation of the piston (132) is stopped and the opening/closing means (135) returns to the original position thereof, the closing means (137) may be positioned in the first transfer pipe (120) so that the transfer of the powder is stopped. On the other hand, the elastic support (139) may be formed on the other side of the opening/closing means (135) to more easily support the operation of the piston (132) due to elasticity.

In addition, by controlling the operation of the piston (132), the areas of the opening hole (136) and the closing means (137) coming into contact with the first transfer pipe (120) may be controllable. Therefore, since the content of powder to be sprayed is controllable, the volume of powder needed for surgery may be sprayed. In this case, since the operation of the piston (132) interworks with a trigger (500) to be described below, the spraying may be performed by setting the range of operation of the piston (132) in advance using a separate opening/closing control button (not illustrated). In this regard, the flow volume control means (130) may be preferably formed and operated as a solenoid valve.

In an embodiment of the present application, the powder spraying device (1) may include the trigger (500) for operating the flow volume control means (130) and the vibrating means (200), and may include a power supply part (600) for supplying power to the flow volume control means (130), the vibrating means (200), and the air supply part (300).

In an embodiment of the present application, as illustrated in FIGS. 1 to 3, the powder spraying device (1) may have a gun shape. Accordingly, the trigger (500) and the power supply part (600) may be located on a handle part. At this time, when the trigger (500) is pushed, the flow volume control means (130) and the vibrating means (200) may be operated. When the trigger (500) is released, the operating of the flow volume control means (130) and the vibrating means (200) may be stopped. That is, since the spraying of the powder is controlled according to whether the flow volume control means (130) is operated, a surgeon may more easily use the powder spraying device (1). In addition, as described above, the air supply part (300) may be operated by pushing the air pump switch (370). That is, when only the air supply part (300) is operated by the air pump switch (370), air may be sprayed to the outside to dry moisture that is present in the powder spraying device (1). Therefore, when the spraying of the powder is performed, a phenomenon in which the powder is agglomerated due to moisture may be prevented. On the other hand, the power supply part (600) for supplying power so as to operated the flow volume control means (130), the vibrating means (200) and the air supply part (300) is required. The power supply part (600) may be inserted into the handle in terms of efficiency of space.

In another embodiment of the present application, referring to FIG. 3, the powder spraying device (1) may not include the flow volume control means (130) and the air pump switch (370) separately. In this case, the trigger (500) may be connected to the air supply part (300) and the vibrating means (200). When the trigger (500) is pushed, the air supply part (300) and the vibrating means (200) may be operated. When the trigger (500) is released, the air supply part (300) and the vibrating means (200) may be stopped. That is, since the spraying of the powder is controlled according to whether the air supply part (300) is operated, a surgeon may more easily use the powder spraying device (1).

A second aspect of the present application provides an operating method of the powder spraying device (1).

The operating method of the powder spraying device (1) according to the first aspect of the present application may include: operating the air supply part (300) to supply air to the second transfer pipe (140); operating the flow volume control means (130) to supply the powder contained in the powder container part (100) to the first transfer pipe (120) and the second transfer pipe (140); and releasing the powder supplied to the second transfer pipe (140) to the outside together with the air.

Although detailed descriptions of parts overlapping the first aspect of the present application are omitted, the descriptions of the first aspect of the present application may be equally applied even when the descriptions thereof are omitted in the second aspect.

Hereinafter, the operating method of the powder spraying device (1) according to the second aspect of the present application will be described step by step in detail.

First, in an embodiment of the present application, the operating method of the powder spraying device (1) may include operating the air supply part (300) to supply air to the second transfer pipe (140).

In an embodiment of the present application, the operation of the air supply part (300) may be performed by pushing the air pump switch (370). This is for drying moisture present in the powder spraying device (1), and may be performed by releasing only air to the outside through the spraying means (400) of the powder spraying device (1).

Next, in an embodiment of the present application, the operating method of the powder spraying device (1) may include: operating the flow volume control means (130) to supply the powder contained in the powder container part (100) to the first transfer pipe (120) and the second transfer pipe (140); and releasing the powder supplied to the second transfer pipe (140) to the outside together with the air.

In an embodiment of the present application, the operation of the flow volume control means (130) may be operated by pushing the trigger (500), and at the same time, the vibration by the vibration means (200) may occur. Specifically, the flow volume control means (130) may operate to position the opening hole (136) in the first transfer pipe (120) so as to supply the powder. That is, by supplying the powder to the second transfer pipe (140) in which the air flow is already formed, the powder and the air may be released through the spraying means (400), and the powder may be sprayed and dosed to a damaged mucosal target for hemostasis.

Thereafter, in an embodiment of the present application, when the hemostasis is completed by using the powder spraying device (1), the operation of trigger (500) may be stopped. At the same time as the stop of the vibration by the vibrating means (200), the piston (132) located in the flow volume control means (130) may be returned to the original position thereof. The supply of the powder may be stopped by positioning the closing means (137) on the first transfer pipe (120).

Thereafter, finally, the air pump switch (370) may be returned to the original position to stop the operation of the air supply part (300), thereby completing treatment for the hemostasis.

The present invention has been described in detail with reference to the preferred embodiments and the drawings, but the scope of the technical idea of the present invention is not limited to these drawings and embodiments. Accordingly, various modifications or equivalents thereof may be possibility in line with the scope of the technical idea of the present invention. Therefore, the scope of the technical idea according to the present invention should be interpreted by the claims, and the technical idea within the equivalents should be interpreted as including in line with the scope of the present invention.

### REFERENCE SIGNS LIST

1: Powder spraying device
100: Powder container part
120: First transfer pipe
130: Flow volume control means
131: Frame
132: Piston
135: Opening/closing means
136: Opening hole
137: Closing means
139: Elastic support
140: Second transfer pipe
200: Vibrating means
300: Air supply part
350: Cover
370: Air pump switch
400: Spraying means
420: Connecting means
500: Trigger
600: Power supply part

### INDUSTRIAL APPLICABILITY

Since a powder spraying device according to the present invention includes a funnel-shaped powder container part and a vibrating means, powder may be easily sprayed without clogging.

In addition, since the powder spraying device according to the present invention includes a flow volume control means to control the flow volume of the supplied powder, the spray volume of the powder needed during surgery may be easily controlled.

1. A powder spraying device (1) comprising:
a powder container part (100) for containing powder;
a first transfer pipe (120) having one side connected to the powder container part (100) to transfer the powder contained in the powder container part (100);
a second transfer pipe (140) connected to the other side of the first transfer pipe (120) to transfer air and the powder transferred from the first transfer pipe (120); and
an air supply part (300) connected to one side of the second transfer pipe (140) to supply air to the second transfer pipe (140),
wherein the first transfer pipe (120) is provided with a flow volume control means (130) to control the flow volume of the powder transferred therein.

2. The powder spraying device (1) of claim 1, wherein the flow volume control means (130) comprises:
an opening/closing means (135) for opening and closing the first transfer pipe (120);
a piston (132) connected to one side of the opening/closing means (135) to perform a forward and backward reciprocating motion of the opening/closing means (135); and
an elastic support (139) connected to the other side of the opening/closing means (135) to support the forward and backward reciprocating motion of the opening/closing means (135).

3. The powder spraying device (1) of claim 2, wherein the opening/closing means (135) has an opening hole (136) formed on one side thereinside.

4. The powder spraying device (1) of claim 2, wherein the flow volume control means (130) further comprises a frame (131) for supporting the first transfer pipe (120), the opening/closing means (135), the piston (132), and the elastic support (139).

5. The powder spraying device (1) of claim 1, wherein the powder container part (100) is formed in a funnel shape in which the diameter thereof decreases toward the first transfer pipe (120).

6. The powder spraying device (1) of claim 1, further comprising an air pump switch (370) for operating the air supply part (300).

7. The powder spraying device (1) of claim 1, wherein a vibrating means (200) is attached to the outer surface of the first transfer pipe (120) or the second transfer pipe (140).

8. The powder spraying device (1) of claim 1, further comprising a spraying means (400) connected to the other side of the second transfer pipe (140) to spray the transferred powder and the air to the outside.

9. The powder spraying device (1) of claim 1, further comprising a trigger (500) for operating the flow volume control means (130).

10. The powder spraying device (1) of claim 1, further comprising a power supply part (600) for operating the air supply part (300) and the flow volume control means (130).

11. A powder spraying device (1) comprising:
a powder container part (100) for containing powder;
a first transfer pipe (120) having one side connected to the powder container part (100) to transfer the powder contained in the powder container part (100);
a second transfer pipe (140) connected to the other side of the first transfer pipe (120) to transfer air and the powder transferred from the first transfer pipe (120); and
an air supply part (300) connected to one side of the second transfer pipe (140) to supply air to the second transfer pipe (140),
wherein the powder container part (100) is formed in a funnel shape in which the diameter thereof decreases toward the first transfer pipe (120).

12. An operating method of the powder spraying device (1) of claim 1, the method comprising:
operating the air supply part (300) to supply air to the second transfer pipe (140);
operating the flow volume control means (130) to supply the powder contained in the powder container part (100) to the first transfer pipe (120) and the second transfer pipe (130); and
releasing the powder supplied to the second transfer pipe (130) to the outside together with the air.
